(19) United States
(12) Patent Application Publication
(10) Pub. No.: US 2020/0074631 A1
(43) Pub. Date: Mar. 5, 2020

(54) Systems And Methods For Identifying Implanted Medical Devices

(71) Applicant: The Board of Regents, The University of Texas System, Austin, TX (US)

(72) Inventors: Luca Giancardo, Houston, TX (US); Eliana E. Bonfante Mejia, Houston, TX (US); Roy F. Riascos Castaneda, Houston, TX (US)

(21) Appl. No.: 16/560,392

(22) Filed: Sep. 4, 2019

Related U.S. Application Data
(60) Provisional application No. 62/726,528, filed on Sep. 4, 2018.

(51) Int. Cl. G06T 7/00; A61B 6/03; A61B 8/00; A61B 5/00

(52) U.S. Cl. CPC G06T 7/0014; A61B 6/032; A61B 6/037; A61B 8/00; A61B 5/0082; A61B 5/7267; G06T 2207/30052; G06T 2207/10104; G06T 2207/10108; G06T 2207/10116; G06T 2207/10132; G06T 2207/20081; G06T 2207/20084; G06T 2207/10081

(57) ABSTRACT

In one embodiment, identifying a medical device that is implanted within a patient includes collecting patient data using a sensor, the patient data comprising data regarding the medical device implanted within the patient, inputting the collected patient data into an implanted medical device identification program that is executed by a computing device, automatically comparing the collected patient data with reference data using the implanted medical device identification program, and automatically identifying one or more known implantable medical devices that are a potential match for the medical device implanted within the patient.

### CROSS-REFERENCE TO RELATED APPLICATION

This application claims priority to co-pending U.S. Provisional Application Ser. No. 62/726,528, filed Sep. 4, 2018, which is hereby incorporated by reference herein in its entirety.

### BACKGROUND

Magnetic resonance imaging (MRI) is a medical imaging technique used to form images of the anatomy and physiological processes of the body. Today, MRI is essential for the evaluation and diagnosis of several medical conditions.

MRI scanners use strong magnetic fields, magnetic field gradients, and radiofrequency waves to generate images. When an individual has an implanted medical device having ferromagnetic properties and undergoes an MRI examination, the device reacts to the magnetic fields and radiofrequency stimulation. This can result in undesirable changes to the device, such as malfunction, displacement, or thermal effects on the tissues surrounding the object.

Because of these potential undesired effects, patients typically undergo a screening process prior to an MRI examination to determine if they have implanted medical devices that could cause problems when MRI is performed or that must be scanned under specific conditions to ensure the safety of the patient and integrity of the implant. If the patient is able to provide the information, he or she can identify it to an appropriate medical professional, for example, using a screening form.

Unfortunately, a significant percentage of patients do not have accurate information about the medical devices implanted in their bodies. In fact, it has been estimated that approximately 5 to 10% of patients referred for MRI do not have conclusive information regarding implanted medical devices. In such circumstances, a radiologist may need to infer the nature of an implanted device by visually inspecting medical images, such as X-rays, of the device. This process can be time consuming and is an inefficient use of radiologist's time. Furthermore, if there are any doubts as to the particular type of device that is implanted within the patient, it may be determined not to perform an MRI examination as a safety precaution. The problem is exacerbated in the emergency room setting, in which case patients may not be able to provide information regarding implantable devices due to an acute medical condition. In cases in which an MRI examination is essential for life-saving procedures, any delay in performing such an examination creates risks to the patient.

In view of the above discussion, it can be appreciated that it would be desirable to have a system and method for quickly and easily identifying medical devices implanted within a patient prior to performing an MRI examination on the patient.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a schematic diagram that illustrates an embodiment of a system and method for identifying an implanted medical device.

FIG. 2 is a block diagram of an embodiment of a computing device shown in FIG. 1.

FIG. 3 is a flow diagram of an embodiment of a method for identifying an implanted medical device.

FIG. 4 is a compilation of X-ray images of programmable cerebrospinal fluid shunt valves (CSF-SVs) used in a study to evaluate a system and method for identifying an implanted medical devices. Each row shows 15 random samples for each of 5 classes of medical devices used in the study. From the top, Row 1: Medtronic Strata II-NSC (n=164); Row 2: Codman-Hakim (n=106); Row 3: Sophysa Polaris SPV (n=82); Row 4: Codman-Certas (n=42); Row 5: Miethke ProGAV (n=22). In parentheses are the number of samples contained in the dataset. The dataset contains most of the shunt valve brands currently used in the United States at the time of filing.

FIG. 5 is a confusion matrix for an Enhanced-Xception Network. Each cell of the matrix shows the ratio of images of a given class (true label) classified into the class indicated by the column (predicted label). A perfect classifier will only have 1.00 in the matrix diagonal.

FIG. 6 is a compilation of X-ray images of 16 samples that were misclassified by the Enhanced-Xception Network. Large foreign objects, low contrast, or acquisition angles not well represented in the dataset are visible.

FIG. 7 includes two images of vena cava filters (VCFs) used in further testing. On the left is a Venatech® filter and on the right is an Optease® filter.

FIG. 8 includes two images of two different patent ductus arteriosus closure devices (PDAs) used in further testing. On the left is a microvascular plug (MVP) and on the right is a PDA clip.

FIG. 9 is a confusion matrix that shows results for the tested CVFs.

FIG. 10 is a confusion matrix that shows results for the tested PDAs.

### DETAILED DESCRIPTION

As described above, it would be desirable to have a system and method for quickly and easily identifying medical devices implanted within a patient prior to performing a magnetic resonance imaging (MRI) examination on the patient. Examples of such systems and methods are described in the disclosure that follows. These systems and methods at least partly automate the implanted medical device identification process. In some embodiments, a system for identifying an implanted device comprises a sensor configured to collect data from the patient that can be used as input for searching for a possible matching implantable medical device, and computing device that executes a computer program that performs the matching process. In some embodiments, the sensor is a medical imaging system or device that is configured to capture images of the implanted medical device that can be input into the computer program, which then performs an image-based identification process in which the acquired images are compared to reference images and/or reference models of known, typically commercially available, implantable medical devices. In some embodiments, the computer program implements a machine-learning algorithm for this purpose. Stored in association with the reference images/models is device identification information, including the type, manufacturer, and model of the medical device, as well as any information that is relevant to performing an MRI examination on a patient in which the medical device is implanted. Accordingly, all information relevant to performing an MRI examination on the patient can be quickly and easily identified, potentially without the need for a radiologist to manually identify the implanted medical device. Not only does this avoid wasting radiologist time, it also speeds up the identification process, which can result in better patient outcomes.

In the following disclosure, various specific embodiments are described. It is to be understood that those embodiments are example implementations of the disclosed inventions and that alternative embodiments are possible. Such alternative embodiments can include hybrid embodiments that comprise features of different embodiments. All such embodiments are intended to fall within the scope of this disclosure.

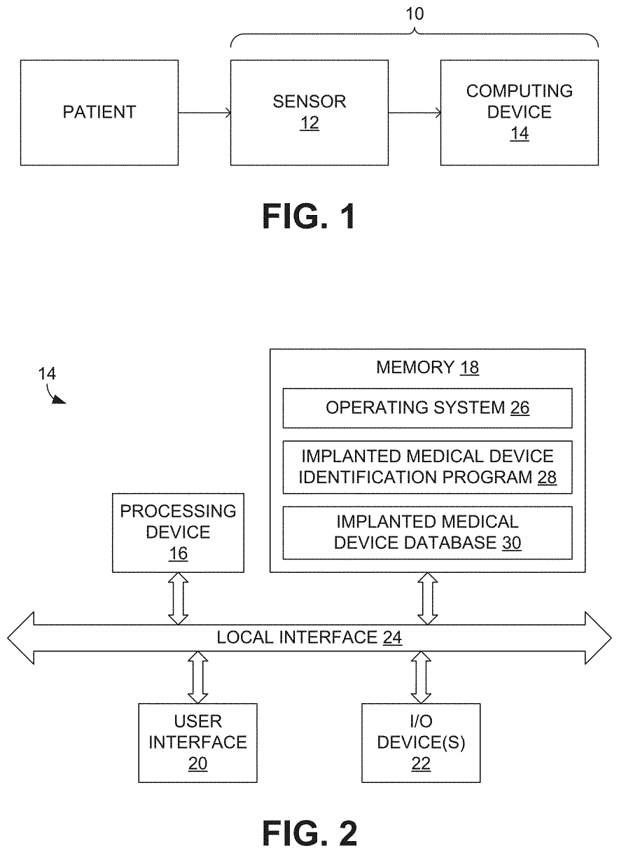

As described above, disclosed are systems and methods for identifying implanted medical devices and information relevant to performing an MRI examination on a patient having such an implant. FIG. 1 provides a high-level overview of the systems and methods. As shown in the figure, a system 10 for identifying implanted medical devices includes a sensor 12 that is configured to acquire data from a patient that can be used as input for a computing device 14 that executes a computer program configured to automatically identify one or more known implantable medical devices that could be a match for the medical device implanted within the patient. As noted above, the sensor 12 can comprise a medical imaging system or device that is configured to capture images of the implanted medical device. Such a system/device can take substantially any form, but examples include X-ray, computed tomography (CT), ultrasound, positron emission tomography (PET), single photon emission computed tomography (SPECT), and medical optical imaging systems/devices. As used herein, “medical optical imaging” refers to imaging modalities that use light for imaging purposes, including optical microscopy, spectroscopy, endoscopy, scanning laser ophthalmoscopy, optical coherence tomography, and interferometric microscopy.

While image data is a good candidate to use as input for implanted medical device identification, it is noted that the sensor 12 is not limited to imaging systems or devices. More generally, the sensor 12 can be configured to collect any patient data that can be used to uniquely identify the implanted medical device. Such data can include, for instance, audio emitted or reflected from the device, electrical signals generated by the device, light patterns reflected off subcutaneously implanted devices, other electromagnetic signals reflected off implanted devices, or any other sensed data that can be used to uniquely identify the medical device. Notably, while some implantable medical devices are specifically configured to provide an indication of the implanted medical device (e.g., using RFID tags or markers), the disclosed systems and methods are configured to identify such information for devices that are not so configured. Therefore, the identification described herein can be achieved even though the implanted medical device comprises no identification means intended for use in identifying the medical device after implantation.

As used herein, the term “identify” is intended to describe identification of one or more implantable medical devices that may be a match for the medical device that is implanted within the patient. In addition to identifying the general type of the implantable medical devices (e.g., pacemaker, valve, stent, joint implant, etc.), identified are the specific makes and models of the implantable medical devices (e.g., manufacturer names and model names of the specific devices). As described below, when this level of specificity can be obtained by using the disclosed systems and methods, the determination as to whether or not to perform the MRI examination (or how to perform it) can be made with greater certainty.

FIG. 2 illustrates an architecture for the computing device 14 shown in FIG. 1. The computing device 10 can comprise any form of computing device having the computing power necessary to quickly and easily identify implantable medical devices based upon input medical images. Example configurations for the computing device 10 include desktop computers, server computers, tablet computers, smart phones, and the like.

As shown in FIG. 2, the example computing device 14 generally comprises a processing device 16, memory 18, a user interface 20, and one or more input/output (I/O) devices 22, each of which is connected to a system bus 24. The processing device 16 can, for example, include a central processing unit (CPU) that is capable of executing computer-executable instructions stored within the memory 18. The memory 16 can include any one of or a combination of volatile memory elements (e.g., RAM, flash, etc.) and nonvolatile memory elements (e.g., hard disk, ROM, etc.). The user interface 20 can comprise one or more devices that can enter user inputs into the computing device 14, such as a keyboard and mouse, as well as one or more devices that can convey information to the user, such as a display. The I/O devices 22 can comprise components that enable the computing device 14 to communicate with other devices, such as a network adapter and a wireless transceiver. While particular components are illustrated in FIG. 1, it is noted that the computing device 14 need not comprise each of these components and that the computing device can comprise other components. For example, in some embodiments, the computing device 14 can further comprise a graphical processing device, such as a graphical processing unit (GPU).

The memory 18 (a non-transitory computer-readable medium) stores programs (software) including an operating system 26 and an implanted medical device identification program 28. The operating system 26 controls the general operation of the computing device 10, while the implanted medical device identification program 28 comprises computer-executable instructions, which may be comprised by one or more algorithms (computer logic), that can be used to identify implantable medical devices from the input data, such as one or more medical images, such as X-ray images, CT images, ultrasound images, PET images, SPECT images, medical optical imaging images, or the like. The memory 18 can further comprise an implanted medical device database 30 that stores reference data, such as reference images and/or reference models, of known implantable medical devices as well as information about the devices that may be relevant to performing an MRI examination on patients having the medical devices implanted within their body. As described below, the reference data can be used in the medical device identification process performed by the implanted medical device identification program 24.

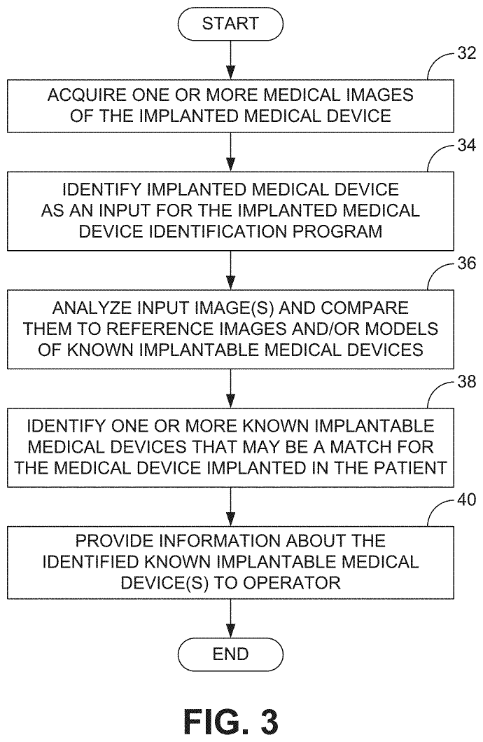

FIG. 3 is a flow diagram of an example method for identifying an implanted medical device that can be performed using a computing device, such as the computing device 14 shown in FIG. 1. In the example of FIG. 3, it is assumed that a patient for whom an MRI examination is desired lacks information about an implanted medical device or is unconscious or otherwise unable to communicate, and that the patient data collected by the sensor 12 is medical image data that is acquired using a medical imagining system or device.

Beginning with block 32 of FIG. 2, one or more medical images of the implanted medical device are acquired. As noted above, the medical images can be obtained using any one of a variety of medical imaging modalities, such as radiography, CT, ultrasound, PET, SPECT, medical optical imaging, and the like. Irrespective of the manner in which the medical images are acquired, an operator can identify an implanted medical device in one or more of the medical images as an input for the medical device identification program, as indicated in block 34. It is this input that the program can use in searching for a potential match. In alternative embodiments, the implanted medical device identification program can be configured to automatically identify the implanted medical device within the one or more images.

It is noted that, in some embodiments, the search functionality of the implanted medical device identification program may be enhanced by receiving textual input from the operator along with the one or more input images (or other data). Such textual input could reflect the operator's professional opinion as the likely type, manufacturer, and/or model of the medical device that is implanted in the patient after having reviewed the collected patient data. Such an input could involve simply selecting one or more options from one or more lists presented to the operator (e.g., a type list, a manufacturer list, and/or a model list), or the input of search terms or phrases by the operator into a suitable dialog box. In the latter case, a natural language processing algorithm can be used that creates an embedding, or vector representation, of the textual query that can be concatenated to the image embedding performed by the implanted medical device identification program. Irrespective of the nature of the operator input, the input can be used in conjunction with the input images (or other data) to narrow the field of potential matches.

With reference next to block 36, the medical device identification program analyzes the input image or images and compares them to reference images and/or reference models of known implantable medical devices stored in the database. In some embodiments, the input images are compared to like types of reference images. For example, CT images of the implanted medical device can be compared to reference CT images of the same device implanted in previous patients. In such a case, the type of image input into the implanted medical device identification program can be manually identified by an operator or automatically identified by the implanted medical device identification program. When such parity is needed, the type of image modality used to create the input images may be made in relation to the types of reference images that are stored in the implanted medical device database. In other embodiments, the implanted medical device identification program is configured to compare not only like types of images but also disparate types of images as well as computer models (e.g., two-dimensional and/or three-dimensional models). In such a case, the implanted medical device identification program can, for example, be configured to compare X-ray images with reference MRI images in order to identify possible matches.

In some embodiments, the implanted medical device identification program analyzes the input image or images using one or more machine-learning algorithms that have been trained to identify possible matches. Example machine-learning algorithms include neural-network algorithms and feature engineering algorithms that are configured to perform image feature extraction and classification to identify possible matches. Specific examples of the operation of such machine-learning algorithms are described below in relation to Examples 1 and 2. In some embodiments, the implanted medical device identification program can also be configured to automatically identify or estimate one or more parameters of the implanted medical device. For example, if the implanted medical device is a device that has different settings that can be selected, such as the flow settings on a shunt valve, those settings can also be determined.

Once the above analysis has been performed, the implanted medical device identification program identifies one or more known implantable medical devices that are potential matches for the medical device that is implanted in the patient, as indicated in block 38. As expressed above, such identification can be achieved even when the implanted medical device comprises no identification means that were provided on the device with the intention of facilitating identification of the medical device after it has been implanted. It is also noted that, even if the implanted medical device identification program cannot narrow the search down to a single match with high probability, receiving an identification of multiple potential matches out of the large universe of medical devices that potentially could be implanted within the patient is exceedingly useful as such information greatly narrows down the field of possible devices and still may enable the operator or other medical professional to determine whether or not an MRI examination is advisable or inadvisable.

Once the implanted medical device identification program has identified one or more potential matches for the medical device implanted within the patient, the program can provide information about the identified implantable medical device(s) to the operator (block 40) to enable that person or another medical professional to make a determination as to whether or not to perform an MRI examination on the patient. As noted above, this information can include not only the type of medical device that is implanted within the patient but also the manufacturer and the model of the medical device, as well as any information about the device that is relevant to a medical professional considering performing an MRI examination on the patient. In some embodiments, this information can include information that was obtained from a safety profile for the particular identified implantable medical devices. Based upon this information, a medical professional can determine whether or not to proceed with the MRI examination.

Experiments were performed to determine the accuracy of prototype implanted medical device identification programs in correctly identifying implanted medical devices from medical images. These experiments are described in the following examples.

### Example 1

A study was conducted to evaluate whether a machine-learning method can distinguish models of cerebrospinal fluid shunt valves (CSF-SVs) from their appearance in clinical X-rays.

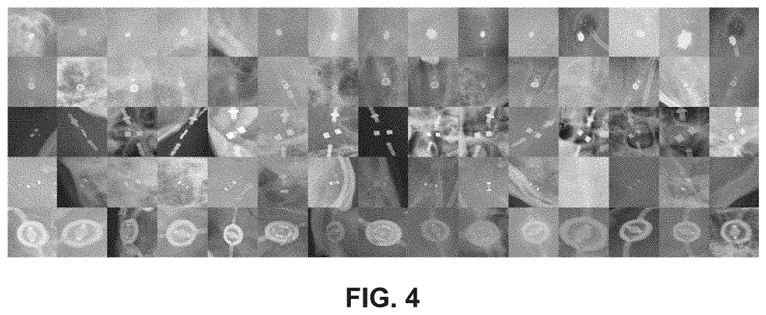

A total of 416 skull X-rays that included a CSF-SV image were collected from the institutional PACS. The images were acquired as a part of a quality improvement project approved by the University of Texas Health Science Center (Houston) Institutional Review Board (IRB). Different versions of the same CSF-SV models where grouped together. The specific five-class grouping was as follows: Codman-Certas® (42), Codman-Hakim® (106), Miethke ProGAV® (22), Sophysa Polaris SPV® (standard/140/400) (82) and Medtronic Strata® (II/NSC) (164). All images were acquired from different subjects and were selected regardless of acquisition perspective or scale. An expert radiologist was asked to select an image window of 300×300 pixels spanning each valve. Such image windows contained valves from different perspectives, scales, and brightness, as well as confounding background objects such as: bone structures, craniotomy hardware, or catheters, as shown in FIG. 4. This dataset simulated a system in which the X-ray operator or radiologist clicks on an implanted device identified in the X-ray images to see the relevant implanted device safety profile.

Four different machine learning-based pipelines were developed and tested. Each pipeline can be broadly split into an image feature extraction and classification phase. During the image feature extraction phase, a compact numerical representation of the image is generated by encoding the visual information into a fixed-size feature vector whose size is significantly less than the number of pixels in the image. These vectors can be created using feature engineering, in which measurements are based on a predetermined set of imaging operators or learned by using representation learning approaches, such as those implemented by deep convolutional neural networks (DCNNs). In the classification phase, feature vectors were used as input for a machine-learning classifier that predicts the most likely valve type.

The images went through the basic pre-processing step of histogram normalization, which ensures that the dynamic range of the image histogram is a value between 0 and 1. Then, two feature extraction pipelines were implemented using validated feature engineering approaches, one based on local binary patterns (LBP) and one based on histogram of oriented gradients (HOG).

LBP features are computed by splitting the image in local windows. Each pixel in the window is compared to its neighbors in order to generate a unique code that represents a characteristic of the texture, such as edges, corners, or flat areas. The histogram of these codes is the actual feature vector that will be used as input for the classification phase. In the experiments, a multiscale LBP approach was implemented using a neighbor radius of 6 and 12 pixels, histograms of 30 bins, and LBP codes invariant to image rotations.

HOG features are based the image gradients, i.e., the direction/intensity of the image edges. The distribution of these image gradients was computed in local windows that are then concatenated to create the final feature vector. In the experiments, local windows (or cells) of 20×20 pixels were used. The implementation provided in the scikit-image Python library were leveraged for both LBP and HOG.

In contrast to feature engineering, representation learning using approaches like DCNN enable the creation of feature vectors that are not based on a predefined set of rules, but rather learned from the data at hand. However, modern DCNNs often require hundreds of thousands of images for a complete training, referred to as end-to-end training. Transfer learning is a strategy where a DCNN is first trained on a large dataset containing images unrelated to the problem of interest and then adapted to a smaller dataset. Transfer learning was successfully used for multiple computational medical imaging problems. In the experiments, transfer learning strategies were adopted with a modern DCNN architecture as a feature vector generator. The Xception network architecture, which is a DCNN inspired by Inception V3 where convolutional filters are depthwise separable, was used. This network is composed of 126 layers for a total of 22,910,480 trainable parameters (or network weights). In the experiments, an Xception network that was pre-trained on the Imagenet dataset (available at www.image-net.org) was used. This dataset contains over 14 million of hand-annotated natural color images for 1,000 classes. The last fully-connected layer of the network was removed and a max-pooling layer was added to generate the feature vector.

The images were pre-processed using the same histogram normalization as discussed above with an additional step. Since the network pre-training was performed on color images, the monochrome intensity value of the X-ray images was artificially replicated into 3 channels, which the network interpreted as RGB. Tissues, bones, and implanted devices in X-ray images have well-defined absorption rates as the X-ray beams traverse matter. Therefore, global thresholds are much more effective in differentiating prominent structures from the background than in natural images. Using this insight, a novel pre-processing strategy was devised that enabled the feeding of more domain relevant information into the network, referred as Enhanced-Xception Network. Using this strategy, an image containing a rough estimation of the foreground objects was created. This image was then placed into the red and blue channels, while the original X-ray image was left untouched in the green channel. The foreground objects were roughly estimated using the Otsu thresholding approach. All DCNNs were implemented using the Keras® (www.keras.io) and Tensorflow® (www.tensorflow.org) Python libraries.

The same classification phase was used for all four feature extraction strategies. The generated feature vector was classified using a linear logistic regression classifier with L1 regularization (the default 1.0 was used as the regularization strength). The model was extended to multiclass using a one-verses-all strategy and the coordinate descent optimizer implemented by LIBLINEAR/scikit-learn was used for training. A stratified 10-fold cross-validation was used to evaluate the performance of the machine learning pipelines. To summarize, the dataset was split into 10 chunks (or folds). Each fold maintained the same class distribution of the complete dataset. One fold was left out as a testing set and the classifier was trained on the remaining 9 folds. This operation was iteratively performed for all folds while ensuring that the classifier was reset at each iteration. This strategy avoided overfitting and enabled robust estimation of the classification performance.

Table 1 shows the classification performance of the four machine learning pipelines. Deep convolutional networks trained with a transfer learning strategy outperformed the two feature engineering methods tested by achieving an accuracy of 95-96% (confidence intervals (CI) [94-97]/[94-98]) versus 53/54% (CI [48-56]/[50-59]). Specifically, the Enhanced-Xception Network performed best for all metrics evaluated (precision: 0.96 CI [0.95-0.98], recall: 0.96 [0.94-0.98]). Precision is also known as positive predictive value, and recall is also known as sensitivity. All confidence intervals were computed using the non-parametric bootstrap procedure, using 1,000 repetitions and reporting the 5th and 95th percentiles.

TABLE 1
Classification Performance of the Four Tested Machine-Learning Pipelines

| | | | Avg. | Avg. | Avg. |
|---|---|---|---|---|---|
| | | Accuracy | Precision | Recall | F1-score |
| Feature Engineering | Local Binary | 53% | 0.52 | 0.53 | 0.52 |
| Methods | Patterns (LBP) | [48-56] | [0.48-0.57] | [0.48-0.58] | [0.48-0.57] |
| | Histogram of | 55% | 0.55 | 55 | 0.51 |
| | Oriented Gradients | [50-59] | [0.50-0.60] | [0.50-0.59] | [0.46-0.56] |
| | (HOG) | | | | |
| Deep Convolutional | Xception | 95% | 0.95 | 0.95 | 0.95 |
| Neural Networks | Network | [94-97] | [0.94-0.97] | 0.94-0.97] | [0.93-0.97] |
| (Transfer Learning) | Enhanced-Xception | 96% | 0.96 | 0.96 | 0.96 |
| | Network | [94-98] | [0.95-0.98] | [0.94-0.98] | [0.94-0.98] |

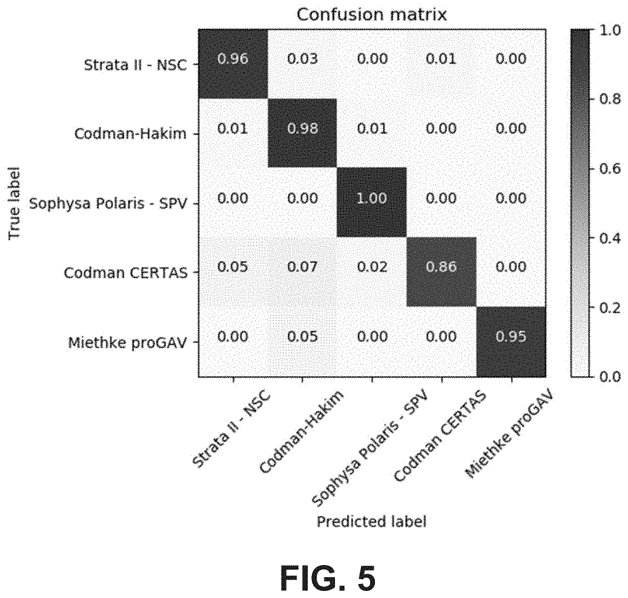

Table 2 shows the performance metric for each valve. All valves were classified with a F1 score equal to or above 0.90, which ranged from 0.99 on the Sophya Polaris SPV class to 0.90 with the Codman-Certas. The F1 score was computed as the harmonic mean of precision and recall. FIG. 5 shows the confusion matrix indicating the correct and wrong classification by true valve class and predicted valve class. No obvious misclassification bias was apparent in the matrix.

TABLE 2
Class-Level Performance Metrics Using
the Enhanced-Xception Network

| | N | Precision | Recall | F1-score |
|---|---|---|---|---|
| Strata II-NSC | 164 | 0.98 | 0.96 | 0.97 |
| Codman-Hakim | 106 | 0.92 | 0.98 | 0.95 |
| Sophysa Polaris SPV | 82 | 0.98 | 1.00 | 0.99 |
| Codman-Certas | 42 | 0.95 | 0.86 | 0.90 |
| Miethke proGAV | 22 | 1.00 | 0.95 | 0.98 |

After training, all four machine pipelines could be run in under 1 second per image on a 3.0 GHz Xeon desktop with a Titan X GPU. Specifically, LBP: ˜0.13 seconds/image; HOG: ˜0.08 seconds/image; Xception Network ˜0.53 seconds/image; Enhanced-Xception Network: ˜0.54 seconds/image.

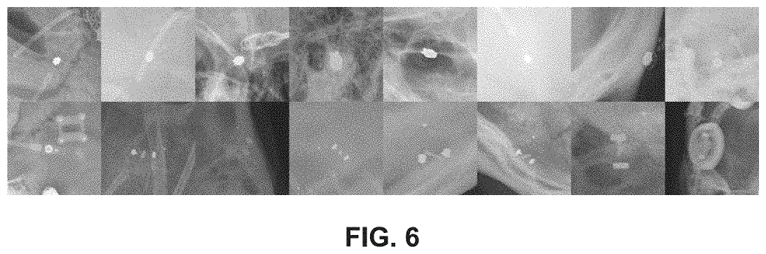

To summarize, an X-ray-based, automatic, implanted medical device identification system for MRI safety was used to test the feasibility of the automatic CSF-SV recognition component. Four different machine learning pipelines were developed and the results indicate that a deep learning based algorithm (Enhanced-Xception Network) can achieve a very high accuracy (96%) in identifying the valves correctly. The 16 (out of 416) images that were misclassified were visually inspected (see FIG. 6). In each case, large foreign objects, low contrast, or acquisition angles not well represented in the dataset were observed. These issues can likely be solved by increasing the dataset size and including an automatic quality assurance algorithm configured to warn the user if the quality of the image is too low for a reliable valve identification. In general, the implanted medical device identification system was able to classify very challenging samples of valves imaged at skewed angles, scales, and locations in the skull.

The best performing algorithm can be run in real time on commercially available hardware, thereby making it possible to integrate it into X-ray machines, hospital picture archiving and communication systems (PACS), or software-as-a-service (SaS) cloud services. Additionally, the disclosed approach does not require any type of protected health information (PHI), thereby drastically reducing security concerns in the translation of the project to clinical practice.

In the envisioned workflow, there may be a human operator visually comparing the various implantable medical devices identified by the implanted medical device identification system according to their image similarity with the input image. Therefore, 100% accuracy is not required.

Although the study focused on CSF-SVs, the disclosed process is not specific to any particular type of implanted medical device and can be readily applied to other implanted medical devices by retraining the system algorithm on other datasets.

### Example 2

The ability of the algorithm referred as Enhanced-Xception Network to identify two other classes of implantable medical devices was also evaluated. Vena cava filters (VCFs) and patent ductus arteriosus closure devices (PDAs) were selected for this study.

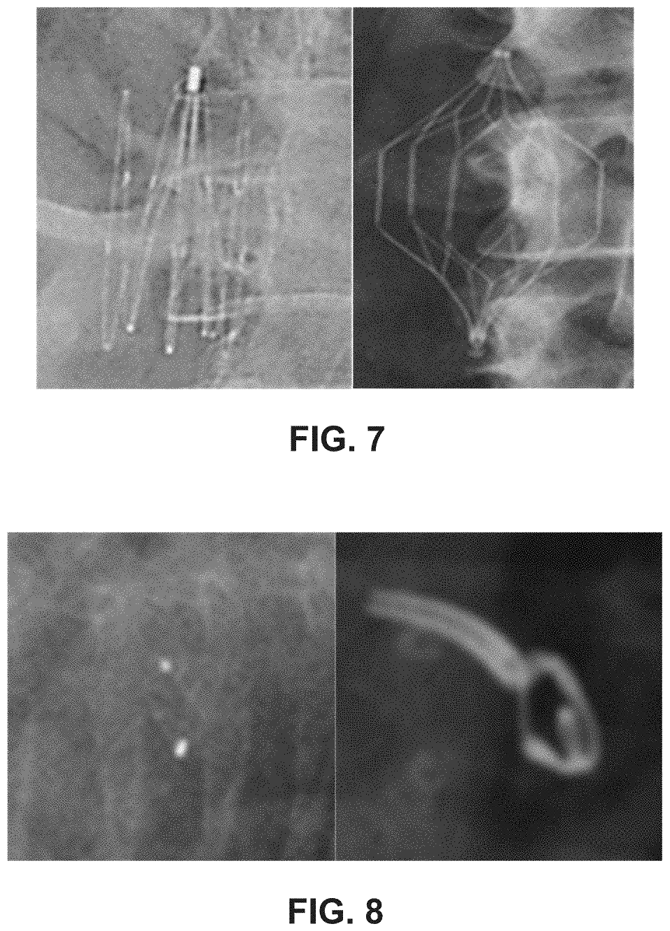

A total of four devices: 2 VCF and 2 PDA were selected. Thirty-three Venatech® filters and 34 Optease® filters were chosen as examples to test the discriminative ability of the algorithm on the VCFs, two of which being illustrated in FIG. 7. Sixty microvascular plugs (MVPs) and 16 PDA clips were chosen as examples to test the discriminative ability of the algorithm on PDAs, two of which being illustrated in FIG. 8.

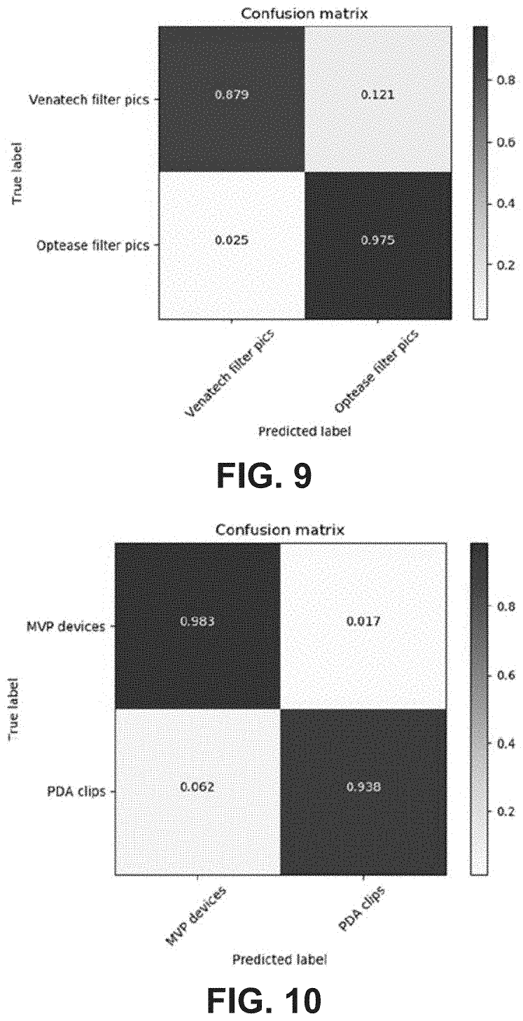

Using the same 10-fold cross-validation approach and Enhanced-Xception Network described in Example 1, the algorithm was trained and tested to distinguish between the two types of CVFs. The following results were obtained (see FIG. 9):
Accuracy: 0.932, [0.879-0.983]
Precision: 0.934, [0.882-0.983]
Recall: 0.932, [0.879-0.983]
F1-score: 0.931, [0.876-0.983]
Area Under the Receiving Operating Characteristic Curve: 0.992 (***p<0.001)

Using the same 10-fold cross validation approach and Enhanced-Xception Network described in Example 1, the algorithm was trained and tested to distinguish between the two types of PDAs. The following results were obtained (see FIG. 10):
Accuracy: 0.974, [0.933-1.000]
Precision: 0.974, [0.933-1.000]
Recall: 0.974, [0.933-1.000]
F1-Score: 0.974, [0.933-1.000]
Area Under the Receiving Operating Characteristic Curve: 0.998 (***p<0.001)

These experiments establish that the pipeline previously developed for shunt valves can be applied with other types of implanted medical devices.

While the disclosed implanted medical device identification systems and methods have been described as being used in the context of determining whether or not to perform an MRI examination on a patient, it is noted that this is just an example application and that other applications may exist. In fact, the implanted medical device identification systems and methods may be used in any context in which a medical device is to be identified from patient data, such as image data. Accordingly, this disclosure is not intended to limit the implanted medical device identification systems and methods to any particular application.

Claimed are:

1. A method for identifying a medical device that is implanted within a patient, the method comprising:
collecting patient data using a sensor, the patient data comprising data regarding the medical device implanted within the patient;
inputting the collected patient data into an implanted medical device identification program that is executed by a computing device;
automatically comparing the collected patient data with reference data using the implanted medical device identification program; and
automatically identifying one or more known implantable medical devices that are a potential match for the medical device implanted within the patient.

2. The method of claim 1, wherein collecting patient data using a sensor comprises acquiring one or more medical images of the implanted medical device using a medical imaging system or device.

3. The method of claim 2, wherein the one or more medical images comprise one or more X-ray images, computed tomography images, ultrasound images, positron emission tomography images, single photon emission computed tomography images, or medical optical imaging images.

4. The method of claim 2, wherein inputting the collected patient data into an implanted medical device identification program comprises inputting the one or more acquired medical images into the implanted medical device identification program.

5. The method of claim 4, wherein automatically comparing the collected patient data with reference data comprises automatically comparing the one or more input medical images with reference medical images or models of known implantable medical devices.

6. The method of claim 5, wherein the reference medical images comprise one or more of X-ray images, computed tomography images, ultrasound images, positron emission tomography images, single photon emission computed tomography images, and medical optical imaging images of known medical devices implanted in one or more previous patients.

7. The method of claim 1, wherein the implanted medical device identification program comprises a machine-learning algorithm that has been trained to automatically identify the one or more known implantable medical devices.

8. The method of claim 7, wherein the machine-learning algorithm comprises a neural-network algorithm or a feature engineering algorithm.

9. The method of claim 7, wherein the machine-learning algorithm comprises a deep convolutional neural network algorithm.

10. The method of claim 9, wherein the deep convolutional neural network algorithm has been trained using a transfer learning strategy.

11. The method of claim 1, further comprising providing information about the one or more known implantable medical devices to a user.

12. The method of claim 11, wherein providing information about the one or more known implantable medical devices comprises providing the types, manufacturers, and models of the one or more known implantable medical devices.

13. The method of claim 12, wherein providing information further comprises providing information relevant to a determination as to whether or not to perform a magnetic resonance imaging examination on the patient.

14. The method of claim 13, wherein the information relevant to a determination as to whether or not to perform a magnetic resonance imaging examination comprises information obtained from safety profiles of the one or more known implantable medical devices.

15. A system for identifying a medical device implanted within a patient, the system comprising:
a computing device configured to execute an implanted medical device identification program, the program being configured to:
receive data collected from a patient;
automatically compare the collected patient data with reference data; and
automatically identify one or more known implantable medical devices that are a potential match for the medical device implanted within the patient.

16. The system of claim 15, further comprising a sensor configured to collect the data from the patient.

17. The system of claim 16, wherein the sensor comprises a medical imaging system or device that is configured to capture medical images of the medical device implanted within the patient.

18. The system of claim 17, wherein the sensor comprises one of an X-ray, computed tomography, ultrasound, positron emission tomography, or single photon emission computed tomography system or device.

19. The system of claim 15, wherein the implanted medical device identification program is configured to compare one or more input images of the implanted medical device with reference medical images or models of known implantable medical devices.

20. The system of claim 19, wherein the reference medical images comprise one or more of X-ray images, computed tomography images, ultrasound images, positron emission tomography images, and single photon emission computed tomography images of known medical devices implanted in one or more previous patients.

21. The system of claim 15, wherein the implanted medical device identification program comprises a machine-learning algorithm that has been trained to automatically identify the one or more known implantable medical devices.

22. The system of claim 21, wherein the machine-learning algorithm comprises a neural-network algorithm or a feature engineering algorithm.

23. The system of claim 22, wherein the machine-learning algorithm comprises a deep convolutional neural network algorithm.

24. The system of claim 23, wherein the deep convolutional neural network algorithm has been trained using a transfer learning strategy.

25. The system of claim 15, wherein the implanted medical device identification program is further configured to provide information about the one or more known implantable medical devices to a user, the information including the types, manufacturers, and models of the one or more known implantable medical devices.

26. The system of claim 25, wherein the implanted medical device identification program is further configured to provide to the user information relevant to a determination as to whether or not to perform a magnetic resonance imaging examination on the patient.

27. A non-transitory computer-readable medium that stores an implanted medical device identification program comprising:
logic configured to receive data collected from a patient;
logic configured to automatically compare the collected patient data with reference data; and
logic configured to automatically identify one or more known implantable medical devices that are a potential match for the medical device implanted within the patient.
